(19) United States
(12) Patent Application Publication
(10) Pub. No.: US 2016/0193133 A1
(43) Pub. Date: Jul. 7, 2016

(54) METHOD FOR THE TREATMENT OF HAIR FROM A COMPOSITION COMPRISING AN ORGANIC MONOACID AND A CELLULOSIC POLYMER

(71) Applicant: Camila BIATO, Rio de Janeiro (BR)

(72) Inventor: Camila BIATO, Rio de Janeiro (BR)

(73) Assignee: L'OREAL, Paris (FR)

(21) Appl. No.: 14/911,550

(22) PCT Filed: Aug. 13, 2013
(86) PCT No.: PCT/BR2013/000305

§ 371 Date: Feb. 11, 2016

(51) Int. Cl. A61K 8/73; A45D 7/06; A61Q 5/06; A61K 8/365; A61K 8/368

(52) U.S. Cl. CPC A61K 8/731; A61K 8/365; A61K 8/368; A61Q 5/06; A45D 7/06; A61K 2800/805; A61K 2800/70

(57) ABSTRACT

The object of the present invention is a method for the treatment of hair, comprising the application to the hair of a composition comprising at least one organic monoacid and at least one cellulosic polymer, and a step of heating the hair at a temperature higher than 100° C. The method according to the invention makes it possible to obtain a solution for treatment of the hair in a way which is lasting and compatible with any type of past or future hair treatment and provides a volume reduction.

The present invention relates to a process for treating hair with a composition comprising an organic monoacid and a cellulosic polymer.

In the hair field, consumers wish to have available compositions which make it possible to introduce a temporary change to their hair and to do this while targeting a good wear property for the effect produced. In general, it is desirable for the change to withstand shampooing operations for a minimum of 15 days, indeed even more, depending on the nature of the said change.

Treatments already exist for modifying the colour or the shape of the hair and also, to some extent, the texture of the hair. One of the known treatments for modifying the texture of the hair consists of the combination of heat and of a formaldehyde-comprising composition. This treatment is especially effective in conferring a better appearance on damaged hair and/or in treating long hair and curly hair.

The heat use can be that of the iron (flat tongs or crimping iron), the temperature of which can generally reach 200° C. or more.

However, there is an increasing desire to avoid the use of such substances, which can prove to be aggressive to the hair and other keratinous substances.

Application WO2011/104282 has thus provided a novel method for semi-permanently smoothing the hair which consists in applying an α-aceto acid solution to the hair for from 15 to 120 minutes, in then drying and, finally, in smoothing the hair with an iron at a temperature of approximately 200° C. The α-aceto acid employed is preferably glyoxylic acid.

However, it has been found that glyoxylic acid may not always be well tolerated, in particular when the scalp is sensitive and/or irritated. Its volatility combined with a hot process may cause problems.

Furthermore, cosmetic formulations of prior art can detrimentally affect the hair and/or detrimentally affect the colour thereof. In point of fact, the desired treatment is very particularly intended to repair artificially dyed hair. It is therefore preferable to avoid the use of active agents capable of introducing a reinforcing effect but while contributing to detrimentally affecting the hair and/or its artificial colour.

The object of the invention is to develop a method for changing the aspect of the hair, such as smoothing and/or straightening hair without detrimental effect on the integrity and/or color of the hair fibres. Another object of the invention is to develop a method for changing the aspect of the hair and resistant to water.

Thus, a subject-matter of the present invention is a method for the treatment of hair, comprising the application to the hair of a composition comprising at least one organic monoacid and at least one cellulosic polymer, and a step of heating the hair at a temperature higher than 100° C.

It has been found that the method according to the invention makes it possible to obtain a solution for treatment of the hair in a way which is lasting and compatible with any type of past or future hair treatment.

In particular, the method according to the invention will not detrimentally affect the integrity of the fibre. In addition, it will improve qualities of use and the mechanical properties of the hair, such as the resistance to moisture, and the sheen, and will reduce volume and frizz. It will also reduce colour modification of hair.

In that which follows, the expression “at least one” is equivalent to the expression “one or more”.

The method of the present invention is carried out with a composition comprising at least one organic mono acid. is chosen from compounds of formula (I), their stereoisomers, their organic or mineral salts or their corresponding solvates:

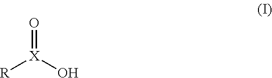

wherein X represents a carbon atom (carboxylic acids), a group P—OH (phosphonic acids), a group P—H (phosphinic acids) or a group S═O (sulfonic acids), and R represents an alkyle, alcenyle, aryle or aralkyle group with a linear, branched or cyclic alkyle group comprising from 1 to 20 carbon atoms, a linear, branched or cyclic alcenyle group comprising from 2 to 20 carbon atoms, an aryle ou aralkyle group comprising from 6 to 20 carbon atoms, these groups being eventually substituted by one or more groups selected from halogen atoms and from the hydroxyl, trifluoromethyl, alcoxy C1-C4 groups.

Among the monoacid of formula (I), mention can be made of glycolic acid (also called hydroxyacetic acid), lactic acid, 1-hydroxy-1-cyclopropanecarboxylic acid, 2-hydroxy-3-butenoic acid, 2-hydroxyisobutyric acid, 3-hydroxybutyric acid, 4-hydroxybutyric acid, 2-hydroxy-n-butyric acid, glyceric acid, 2-hydroxy-3-methylbutyric acid, 2-hydroxy-2-methylbutyric acid, 2-hydroxypentanoic acid, 3-hydroxy pentanoic acid, 4-hydroxypentanoic acid, 2-hydroxyvaleric acid, 1-hydroxycyclohexanecarboxylic acid, mandelic acid, 2-hydroxy-3-methylvaleric acid, 2-trifluoromethyl-2-hydroxypropionic acid, hexahydroxymandelic acid, 2-hydroxyoctanoic acid, 3-phenylactic acid, 3-hydroxymandelic acid, 4-hydroxymandelic acid, 2-hydroxynonanoic acid, 3-methoxymandelic acid, 4-methoxymandelic acid, 3-(4-hydroxyphenyl)lactic acid, □-chlorolactic acid, 1-cyclopentanol-1-carboxylic acid, 1,2-dihydroxycyclobutanecarboxylic acid, 2-ethyl-2-hydroxybutyric acid, □-hydroxyisocaproic acid, □-hydroxycaproic acid, 2-hydroxy-3,3-dimethylbutyric acid, gluconic acid, salicylic acid, 2-hydroxyhexanoic acid, 3-hydroxyhexanoic acid, 4-hydroxyhexanoic acid, 5-hydroxyhexanoic acid, 6-hydroxyhexanoic acid, hydroxypropyl sulfonic acid, methane sulfonic acid, methane phosphonic acid, methyl phosphinic acid and the stereoisomers, organic or mineral salts and solvates thereof.

Particularly preferred monoacid of formula (I) are selected from: glycolic acid, lactic acid, glyceric acid, gluconic acid, salicylic acid, 2-hydroxyisobutyric acid, 3-hydroxybutyric acid, 4-hydroxybutyric acid, 2-hydroxy-n-butyric acid, 2-hydroxyhexanoic acid, 3-hydroxyhexanoic acid, 4-hydroxyhexanoic acid, 5-hydroxyhexanoic acid, 6-hydroxyhexanoic acid, 2-hydroxypentanoic acid, 3-hydroxypentanoic acid, 4-hydroxypentanoic acid, hydroxypropylsulfonic acid), their stereoisomers, their organic or mineral salts or their corresponding solvates.

In a preferred embodiment, the monoacid is a monocarboxylic acid. In this embodiment, moncarboxylic acids are preferably substituted on R radical by at least one hydroxyle group. According to this preference R radical is particularly an alkyle, alcenyle, aryle or aralkyle group with a linear, branched or cyclic alkyle group comprising from 1 to 20 carbon atoms, a linear, branched or cyclic alcenyle group comprising from 2 to 20 carbon atoms, an aryle ou aralkyle group comprising from 6 to 20 carbon atoms, these groups being substituted by at least one hydroxyle group being preferably located in position alpha or beta from the carboy group and these groups being eventually substituted by one or more other groups selected from halogen atoms and from the groups trifluoromethyle, alcoxy C1-C4.

According to this preference, monoacids of formula (I) are selected from glycolic acid, lactic acid, glyceric acid, gluconic acid, salicylic acid, 2-hydroxyisobutyric acid, 2-hydroxy-n-butyric acid, 2-hydroxyhexanoic acid, 2-hydroxypentanoic acid their stereoisomers, their organic or mineral salts or their corresponding solvates.

More preferably monoacids of formula (I) are selected from glycolic acid, lactic acid, glyceric acid, gluconic acid, salicylic acid, their stereoisomers, their organic or mineral salts or their corresponding solvates.

According to a specific embodiment, the method of the invention is carried out with a composition comprising at least two organic mono acids of formula (I) and particularly with at least two mono carboxylic acids of formula (I).

According to a specific embodiment, the method of the invention is carried out with a composition comprising at least glycolic acid with preferably at least one additional monocarboxylic acid of formula (I). Preferably, the composition contains glycolic acid and salicylic acid.

The amount of monoacids, their stereoisomers, their organic or mineral salts or their corresponding solvates in the composition useful in the method of the present invention varies largely. However, monoacids, their stereoisomers, their organic or mineral salts or their corresponding solvates are preferably present in the composition at a concentration ranging from 0.01% to 50% by weight, in particular from 0.1% to 20% by weight and preferably from 1% to 5% by weight, with respect to the total weight of the composition.

The composition useful in the method of the invention contains a cellulosic polymer.

The cellulose polymers may be anionic, cationic, amphoteric or non-ionic polymers.

The term “cellulose” polymer means, according to the invention, any polysaccharide compound having in its structure sequences of glucose residues linked together via β-1,4 linkages; in addition to unsubstituted celluloses, the cellulose derivatives may be anionic, cationic, amphoteric or non-ionic. Thus, the cellulose polymers of the invention can be chosen from unsubstituted celluloses, including in a microcrystalline form, and cellulose ethers. Among these cellulose polymers, cellulose ethers, cellulose esters and cellulose ester ethers are distinguished. Among the cellulose esters are inorganic esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

According to one particular embodiment of the invention, the cellulose polymer(s) are non-associative.

The “non-associative” cellulose polymers of the invention are cellulose polymers which do not comprise a fatty chain, i.e. which preferably do not comprise a C₁₀-C₃₀ chain in their structure.

According to a first variant, the cellulose polymer(s) are non-ionic. Mention may be made of (C₁-C₄)alkylcelluloses such as methylcelluloses and ethylcelluloses for example Ethocel standard 100 Premium from Dow Chemical; (poly)hydroxy(C₁-C₄)alkylcelluloses such as hydroxymethylcelluloses, hydroxyethylcelluloses for example Natrosol 250 HHR provided by Aqualon and hydroxypropylcelluloses for example Klucel EF from Aqualon; mixed (poly)hydroxy(C₁-C₄)alkyl-(C₁-C₄)alkylcelluloses such as hydroxypropylmethylcelluloses for example Methocel E4M from Dow Chemical; hydroxyethylmethylcelluloses, hydroxyethylethylcelluloses for example Bermocoll E 481 FQ from Akzo Noble and hydroxybutylmethylcelluloses.

According to a second variant, the cellulose polymer(s) are anionic. Among the anionic cellulose ethers, mention may be made of (poly)carboxy(C₁-C₄)alkylcelluloses and salts thereof. Examples that may be mentioned include carboxymethylcelluloses, carboxymethylmethylcelluloses for example Blanose 7M from the company Aqualon and carboxymethylhydroxyethylcelluloses, and the sodium salts thereof.

According to a third variant, the cellulose polymer(s) are cationic. Among the cationic cellulose, mention may be made of cationic cellulose derivatives such as cellulose copolymers or cellulose derivatives grafted with a water-soluble quaternary ammonium monomer, and described in particular in patent U.S. Pat. No. 4,131,576, such as (poly)hydroxy(C₁-C₄)alkyl celluloses, for instance hydroxymethyl-, hydroxyethyl- or hydroxypropylcelluloses grafted in particular with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

According to one other particular embodiment of the invention, the cellulose polymer(s) are associative.

The “associative” cellulose polymers of the invention are cellulose polymers which comprises a fatty chain, i.e. which preferably comprises a C₁₀-C₃₀ chain in their structure.

The associative cellulosic polymers may be cationic. Among these cationic associative cellulosic polymers preferred are quaternized (poly)hydroxyethylcelluloses modified by groups comprising at least one fatty chain, such as alkyl, arylalkyl or alkylaryl groups comprising at least 8 carbon atoms, or mixtures of these. The alkyl radicals carried by the quaternized celluloses orhydroxyethylcelluloses above preferably comprise from 8 to 30 carbon atoms. The arylradicals preferably denote phenyl, benzyl, naphthyl or anthryl groups. There may be indicated, as examples of quaternized alkylhydroxyethylcelluloses having C8-C30 fatty chains, the products Quatrisoft LM 200â, Quatrisoft LM-X 529-18-Aâ, Quatrisoft LM-X 529-18-Bâ (C12 alkyl) and Quatrisoft LM-X 529-8â (C18 alkyl), sold by Aqualon, the products Crodacel QMâ, Crodacel QLâ (C12 alkyl) and Crodacel QSâ (C18 alkyl), sold by Croda and the product Softcat SL 100â sold by Aqualon.

The associative cellulosic polymers may be non-ionic. Among these non-ionic associative cellulosic polymers preferred are celluloses or their derivatives, modified by groups comprising at least one fattychain, such as alkyl, arylalkyl or alkylaryl groups or their mixtures, where the alkyl groups are C8-C30 alkyl groups and in particular: nonionic alkylhydroxyethylcelluloses, such as the products Natrosol Plus Grade 330 CS and Polysurf 67 (C16 alkyl) sold by Aqualon; nonionic nonoxynyl hydroxyethylcelluloses, such as the product Amercell HM-1500 sold by Amerchol; nonionic alkylcelluloses, such as the product Bermocoll EHM 100 sold by Berol Nobel.

The cellulose polymers are preferably non-ionic and free of fatty chain (non associative non ionic alkylcellulose polymer). Preferably, the cellulose polymer(s) useful in the invention are chosen from non ionic cellulose ethers such as (C₁-C₄)alkylcelluloses, (poly)hydroxy(C₁-C₄)alkylcelluloses (poly)hydroxy(C₁-C₄)alkyl-(C₁-C₄)alkylcelluloses such as hydroxypropylmethylcelluloses hydroxyethylmethylcelluloses hydroxyethylethylcelluloses; hyd roxybutylmethylcelluloses hydroxymethylcelluloses; hydroxyethylcelluloses and hydroxypropylcelluloses.

The cellulose polymer(s) may be present in the composition useful in the invention in an amount ranging from 0.05% to 10% by weight, in particular from 0.1% to 5% by weight and preferably from 0.2% to 3% by weight relative to the total weight of the composition.

The composition used in the method of the invention preferably does not contain a colouring agent or reducing agent.

The term “colouring agents” is understood to mean, according to the present invention, agents for colouring keratinous fibres, such as direct dyes, pigments or oxidation dye precursors (bases and couplers). If they are present, their content does not exceed 0.001% by weight, with respect to the total weight of the composition. This is because, at such content, only the composition would be dyed, that is to say that an effect of dyeing the keratinous fibres would not be observed.

It should be remembered that oxidation dye precursors, oxidation bases and couplers are colourless or only slightly coloured compounds which, by a condensation reaction in the presence of an oxidizing agent, give a coloured entity. With regard to direct dyes, these compounds are coloured and exhibit a degree of affinity for the keratinous fibres.

The term “reducing agent” is understood to mean, according to the present invention, an agent capable of reducing the disulfide bonds of the hair, such as the compounds chosen from thiols, alkali metal sulfites, hydrides or phosphines.

The compositions used according to the invention can be provided in any formulation form conventionally used and in particular in the form of an aqueous, alcoholic or aqueous/alcoholic solution or suspension or oily solution or suspension; of a solution or a dispersion of the lotion or serum type; of an emulsion, in particular having a liquid or semi-liquid consistency, of the O/W, W/O or multiple type; of a suspension or emulsion having a soft consistency of (O/W) or (W/O) cream type; of an aqueous or anhydrous gel, or of any other cosmetic form.

The composition can comprise one or more organic solvents, for example water-soluble solvents such as C₁-C₇ alcohols; mention may be made of aliphatic C₁-C₇ monoalcohols, aromatic C₆-C₇ monoalcohols, C₃-C₇ polyols or C₃-C₇ polyol ethers, which can be employed alone or as a mixture with water.

The compositions of the invention can be aqueous or anhydrous. Advantageously, the composition comprises from 20 to 95% of water based on the total weight of the composition.

More preferably the composition is a hydro alcoholic composition

The composition used in the method of the invention can additionally comprise at least one additional ingredient for example chosen from oils; solid fatty substances such as fatty alcohol, fatty ester; non-ionic surfactants, surfactants; sunscreens; moisturizing agents; antidandruff agents; antioxidants; chelating agents; pearlescent and opacifying agents; plasticizing or coalescing agents; fillers; silicones an particularly aminosilicones; thickeners other than cellulosic polymers; gelling agents; emulsifiers; conditioning or styling polymers; fragrances; basifying or acidifying agents; silanes; or crosslinking agents.

Depending on their nature and the purpose of the composition, the additional ingredients can be present in an amount which can be easily determined by a person skilled in the art.

In the method of the invention, the composition exhibits preferably an acidic pH, more preferably less than 4. In a preferred embodiment the pH can vary from 1 to 3, preferably from 1.5 to 3, better from 1.7 to 3.

pH can be adjusted with acidic agents other than organic monoacids of the invention such as mineral acids as chlorhydric acid or phosphoric acid, or with basic agents such as mineral basic agents as ammonia, carbonates, bicarbonates, hydroxides or organic basic agents such as alcanolamines.

These compositions can be packaged in pump-action sprays or in aerosol containers, in order to provide for application of the composition in the vaporized (lacquer) form or in the form of a mousse. Such packaging forms are indicated, for example, when it is desired to obtain a spray or a mousse, for the treatment of the hair. In these cases, the composition preferably comprises at least one propellant.

The method according to the invention can be provided in the form of a hair product, in particular a hair care, styling or shaping product. The method especially finds advantageous applications for the retention of the hairstyle or the shaping of the hair, or the volume and frizz reduction. Additional advantages are for the conditioning, shaping, reinforcing and/or repairing of the hair, in particular for improving the disentangling, smoothing, combability, manageability and softness of the hair.

The method of the invention comprises a heating step at a temperature higher than 150° C., preferably ranging from 150 to 250° C. The heating step can be carried out with irons such as flat irons, flat tongs or a crimping iron capable of raising the temperature to a value ranging from 150° C. to 250° C.

According to a specific embodiment, the composition is applied on dried hair. After a posing time, usually of at least 10 min, preferably between 15 and 45 min, more preferably around 30 min, the hair are brushed and then straightened with a flat iron at a temperature of at least 150° C., preferably ranging from 200 to 250° C. Then the hair are rinsed with water and dried.

Before the application of the composition, the hair can be shampooed and dried.

The method of the invention is preferably used without a deformation step using reducing agent or a composition with pH over 10.

The following examples serve to illustrate the invention without, however, exhibiting a limiting nature.

### EXAMPLES

The following composition was prepared at the time of use (given in weight %), pH of the composition being adjusted to 2.2±0.2:

| | |
|---|---|
| METHYL HYDROXYETHYLCELLULOSE sold under | 1% |
| the name Structure ®cell 8000 M by AKZONOBEL | |
| GLYCOLIC ACID in aqueous solution (70%) sold under | 10% |
| the name GLYPURE 70 by DUPONT | |
| SALICYLIC ACID sold by Novacyl | 2% |
| ETHANOL | 30% |
| SODIUM HYDROXIDE | 0.027% |
| AMODIMETHICONE (and) TRIDECETH-5 (and) | 2% |
| TRIDECETH-10 sold under the name DC(R)2-2899 | |
| by DOW CORNING | |
| AMODIMETHICONE (and) TRIDECETH-6 (and) | 2% |
| CETRIMONIUM CHLORIDE sold under the name | |
| Belsil ADM by Wacker | |
| WATER | QSP |

The above composition was applied on shampooed and blow-dried naturally curled hair. After having left the composition on hair for 30 min, the hair were brushed and then heated with a flat iron at a temperature from 200-250° C. Then the hair was rinsed.

By using the method of the invention, the hair thus treated exhibit a substantial reduction of volume

1: A method for treating hair, comprising:
applying to hair a composition comprising an organic monoacid and a cellulosic polymer, and
heating the hair at a temperature higher than 100° C.

2: The method of claim 1, wherein the organic monoacid is a compound of formula (I), a stereoisomer of the compound, an organic or mineral salt of the compound, or a solvate of the compound: 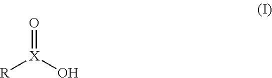
wherein X represents a carbon atom, a group P—OH, a group P—H or a group S═O and R represents an alkyl, alkenyl, aryl or aralkyl group, optionally substituted by one or more groups selected from the group consisting of a halogen atom, a hydroxyl group, a trifluoromethyl group, and a C1-C4 alkoxy group.

3: The method of claim 1, wherein the organic monoacid is a carboxylic acid.

4: The method of claim 2, wherein in the formula (I), X is a carbon atom and R is a linear, branched or cyclic alkyl group comprising from 1 to 20 carbon atoms, a linear, branched or cyclic alkenyl group comprising from 2 to 20 carbon atoms, or an aryl or aralkyl group comprising from 6 to 20 carbon atoms, wherein the alkyl, alkenyl, aryl or aralkyl group is substituted by at least one hydroxyl group.

5: The method of claim 1, wherein the organic monoacid comprises glycolic acid, lactic acid, glyceric acid, gluconic acid, salicylic acid, 2-hydroxyisobutyric acid, 2-hydroxy-n-butyric acid, 2-hydroxyhexanoic acid, 2-hydroxypentanoic acid, a stereoisomer thereof, an organic or mineral salt thereof, or a solvate thereof.

6: The method of claim 2, wherein the composition comprises at least two of the organic monoacids of formula (I).

7: The method of claim 2, wherein the composition comprises glycolic acid and an additional monocarboxylic acid of formula (I).

8: The method of claim 1, wherein the organic monoacid is present in the composition in an amount from 0.1 to 50% by weight, with respect to a total weight of the composition.

9: The method of claim 1, wherein the composition comprises at least one cellulosic polymer selected from the group consisting of an anionic cellulosic polymer, a cationic cellulosic polymer, an amphoteric cellulosic polymer, and a non-ionic cellulosic polymer.

10: The method of claim 1, wherein the composition comprises at least one cellulosic polymer selected from the group consisting of an associative cellulosic polymer and a non-associative cellulosic polymer.

11: The method of claim 1, wherein the composition comprises at least one non-associative non-ionic cellulosic polymer selected from the group consisting of a (C₁-C₄)alkylcellulose, a (poly)hydroxy(C₁-C₄)alkylcellulose, and a (poly)hydroxy(C₁-C₄)alkyl-(C₁-C₄)alkylcellulose.

12: The method of claim 1, wherein the cellulosic polymer is selected from the group consisting of a hydroxypropylmethylcellulose, a hydroxyethylmethylcellulose, a hydroxyethylethylcellulose, a hydroxybutylmethylcellulose, a hydroxymethylcellulose, a hydroxyethylcellulose and a hydroxypropylcellulose.

13: The method of claim 1, wherein the cellulosic polymer is present in the composition in an amount ranging from 0.05% to 10% by weight, relative to a total weight of the composition.

14: The method of claim 1, wherein the composition is hydro alcoholic.

15: The method of claim 1, wherein the composition has an acidic pH.

16: The method of claim 1, wherein the heating is carried out at a temperature from 150 to 250° C.

17: The method of claim 1, wherein the composition is applied on dried hair, and after a posing time of at least 10 min, the hair is brushed and then straightened with a flat iron at a temperature of at least 150° C.

18. (canceled)
